(19) United States
(12) Patent Application Publication
(10) Pub. No.: US 2013/0158524 A1
(43) Pub. Date: Jun. 20, 2013

(54) Releasing Device for Detaching a Medical Implant from a Catheter and a Catheter having a Releasing Device, and Method for Producing a Clamping Body of a Releasing Device and Method for Clamping an Implant in such a Clamping Body

(71) Applicant: BIOTRONIK AG, Buelach (CH)

(72) Inventor: Amir Fargahi, Buelach (CH)

(73) Assignee: BIOTRONIK AG, Buelach (CH)

(21) Appl. No.: 13/693,936

(22) Filed: Dec. 4, 2012

Related U.S. Application Data
(60) Provisional application No. 61/570,312, filed on Dec. 14, 2011.

(51) Int. Cl. A61B 19/00

(52) U.S. Cl. CPC A61B 19/00
USPC 606/1

(57) ABSTRACT

A releasing device (100) for detaching a medical implant (105) from an insertion device (110), wherein the implant (105) can be released by a relative movement between a first and a second insertion element (52, 54), the releasing device comprising a clamping body (10) for clamping the implant (105) in the insertion device (110), with a proximal end (12) which, in the state of use, is remote from a distal end (120) of the insertion device (110) and a distal end (14) which, in the state of use, faces toward the distal end (120) of the insertion device (110), wherein in the clamping state, the clamping body (10) encloses the implant (105) at least at one place (106) in a cap-like manner.

### CROSS REFERENCE TO RELATED APPLICATIONS

This application claims benefit of priority to U.S. provisional patent application Ser. No. 61/570,312, filed Dec. 14, 2011; the contents of which are herein incorporated by reference in their entirety.

### TECHNICAL FIELD

The invention relates to a releasing device for detaching a medical implant from a catheter and to a catheter having a releasing device for releasing a medical implant for implantation in an animal and/or human body, and to a method for producing a clamping body of a releasing device and to a method for clamping an implant in such a clamping body.

### BACKGROUND

In the medical field, implants are frequently used which, for fulfilling substituting functions, are permanently, or at least for an extended period, introduced in an animal and/or human body. To be mentioned here are, for example, heart pacemakers, brain pacemakers for Parkinson patients, heart implants, cochlear implants, retina implants, dental implants, implants for joint replacement, vascular prostheses or stents.

Prior to inserting into the body, implants are connected to catheters and have to be attached in such a manner that they can be accurately placed and released by the catheter in a defined manner at the site of use and without complications. For this purpose it is known, for example, to equip the implant with eyes which interact with hooks on the catheter and to attach the implant to the catheter in this manner.

### SUMMARY

It is an object of the invention to provide a releasing device by means of which connecting an implant to an insertion device is possible in a simple and user-friendly manner and by means of which a highly precise and selective release of an implant can be carried out.

A further object is to be seen in the provision of a corresponding insertion device.

Furthermore, another object is to provide a method for producing a clamping body of such a releasing device.

Moreover, a further object is given by the provision of a method for clamping an implant by means of such a clamping body of the corresponding releasing device.

Provided is a releasing device for detaching a medical implant from an insertion device wherein the implant can be released by a relative movement between a first and a second insertion element. The releasing device comprises a clamping body for clamping the implant in the insertion device with a proximal end which, in the state of use, is remote from a distal end of the insertion device, and with a distal end which, in the state of use, faces toward the distal end of the insertion device, wherein the clamping body, in the clamping state, encloses the implant at least at one place in a cap-like manner.

With the configuration according to the invention, a releasing device can be provided in which the implant is securely retained or is securely fastened on an insertion element of the insertion device such as, for example, an inner shaft. The clamping body further allows a compact and simple construction of the releasing device. In addition, the releasing device provides for a simple handling and mounting of the implant on the insertion device, for example, a catheter, in the preparation laboratory. Furthermore, releasing the implant is reliable and fast. Further, a risk of deforming the implant and a resulting blocking of the releasing device, as it occurs in case of devices from the prior art which operate with hooks and eyes, can be eliminated.

In this connection, a “clamping body” is to be understood as a body which, by means of a clamping effect and/or a force fit, secures another element, in particular the implant, on a component of the insertion device, in particular on an insertion element or the inner shaft, in a fixed position. Here, the clamping body itself can exert the clamping effect, or it can interact with at least one further element, in particular with the insertion device such as, for example, an insertion element or the inner shaft and/or an outer shaft of the insertion device. In addition, the clamping body can have a holding effect that is provided separately from the clamping effect, for example, through a material property. Here, a “clamping state” represents a state in which the implant is captively retained in the insertion device. “Cap-like” is to be understood here such that the clamping body encloses the at least one place of the implant in the circumferential direction of the implant and in at least one region which extends along a plane which is arranged perpendicular to an axial direction of the implant.

Furthermore, it is provided that the clamping body has at least two sheath parts wherein the cap body can be and/or is advantageously adapted to a potentially variable geometry of the implant. A “sheath part” is to be understood here as a portion of the cap body which, in the clamping state, extends at least over a region of a circumference of the implant and/or encloses the same at least partially. The sheath parts are preferably arranged at the same end of the clamping body, advantageously, in the mounted state of the clamping body in the insertion device at the distal end. Furthermore, one end of the clamping body can be widened and/or expanded relative to the other end with respect to its extension and/or its diameter. This can be carried out with any method considered by the person skilled in the art as being applicable and/or depends in particular on a material of the clamping body. It is preferred to widen the end by means of heat supply. Preferably, the distal end is widened, whereby also the sheath parts or their distal ends are widened. In principle, the clamping body could also be configured elastically, and the end could be elastically widened in the clamping state.

Due to the widened embodiment of the ends of the sheath parts, the latter can overlap at their ends when connecting said ends in a direction of their extension and/or in the circumferential direction, whereby clamping the implant is facilitated. This is in particular the case if the overlapping regions of the ends are from a material with high static friction. Due to the partial overlap of the ends of the sheath parts, an initial extension and/or an initial diameter of the expanded end which were or was present prior to the expansion of the same and which correspond or corresponds to an extension or a diameter, respectively, of the opposing end, can be set in a constructionally simple manner. Hereby, a clamping body is provided which, on the one hand, exerts a high clamping effect and, on the other, has a homogenous contour in the axial direction. In addition, this allows a compact construction of the insertion device.

In an advantageous configuration it is proposed that in the clamping state, the two expanded, at least partially overlapping, sheath parts of the clamping body enclose at least a proximal end of the implant, whereby the clamping body is arranged toward the user or toward the proximal end of the insertion device and thereby, for example, provides in addition a contact surface for a sliding movement of the implant in the insertion device. Furthermore, a proximal end of the clamping body can also have a stopper which serves for limiting a movement of the implant in the direction of a proximal end of the insertion device, which would result in a blocking of the implant.

Advantageously, an interaction in the clamping state between a holding force of an insertion element and a holding force of the clamping body keeps the implant in position, whereby a sliding of the implant out of the releasing device or the clamping device is prevented. Furthermore, it is advantageous if the clamping body can be opened due to force applied by the clamping body itself. By automatically opening, a high precision when positioning the implant can be achieved, instead of using hooks and eyes as in the prior art. According to a preferred configuration, the clamping body can be opened due to a radial force of the implant, whereby the releasing device can be implemented while saving components, required space, assembly efforts, and costs because a separate releasing means is not required. In addition, this allows an easy and controlled usability of the releasing device.

In a further configuration of the invention, it is proposed that the clamping body has a feed-through for one of the insertion elements. This allows a compact arrangement which stabilizes and protects the insertion element fed through. If the insertion device is a catheter, the respective insertion element can be an inner shaft of the catheter.

It is further proposed that the clamping body has a material with high static friction so as to keep the implant in position in the clamping state, whereby a fixation of the implant can be carried out in a constructionally simple manner. Here, the static friction between the clamping body and the proximal end of the implant is generated in the insertion device. The material can be any material considered by the person skilled in the art as being useful, such as, in particular, a polymer and in particular a material selected from the group consisting of polyamide, polyester, polyether block amide, silicone, polyurethane. Hereby, the clamping body can in particular be implemented with a low weight. A particularly reliable positioning of the implant in the insertion device can be advantageously achieved due to its high static friction if the material is, e.g., a polyether block amide such as PEBAX, of the company Arkema. Here, all degrees of hardness can be used. In a preferred implementation, the clamping body is made in a mono-layer design. For this, in particular materials such as polyamides, polyester, or polyether block amides such as, preferably, PEBAX, can be used.

In an alternative configuration, the clamping body is implemented in a multi-layer design or a so-called multi-layer co-extruded design. Here, the clamping body has at least two layers which are arranged in the radial direction one behind the other. The radially outer layer or the outer layer is selected here from a material with low friction, and the radially inner layer or the inner layer is selected from a material with higher friction. Here, the outer layer preferably has a material selected from the group consisting of perfluoroethylene propylene (FEP), high density polyethylene (HDPE), polytetrafluoroethylene (PTFE, Teflon), or polyamide (PA), in particular PA-6, -6.6, -6.10, -6.12, -11, -12. The inner layer preferably comprises here a material selected from the group consisting of polyamide, polyester, polyether block amide (PEBAX), silicone, polyurethane, (PUR).

For a good contact between the two layers, a further middle layer can be provided. The middle layer is preferably made from a material that is suitable as a bonding agent, i.e., can generate an adhesion between outer and inner layer. The middle layer preferably comprises the material linear low density polyethylene (LLDP). Furthermore, a coating for reducing the friction can be used on the radially outer layer or the outer layer. This coating can be hydrophobic or hydrophilic and can consist of any material considered by the person skilled in the art as being useful.

According to a further aspect of the invention, an insertion device for inserting a medical implant is proposed which can be released by a relative movement between a first and a second insertion element, the insertion device comprising a releasing device for detaching the medical implant, including a clamping device for clamping the implant in the insertion device, with a proximal end which, in the state of use, is remote from a distal end of the insertion device, and a distal end which, in the state of use, faces toward the distal end of the insertion device, wherein the clamping body encloses the implant in the clamping state at least at one place in a cap-like manner.

With the configuration according to the invention, an insertion device can be provided in which the implant is securely retained or is securely attached on the inner shaft of the insertion device. The clamping body further allows a compact and simple construction of the insertion device. In addition, the implant can be mounted in a simple manner on the insertion device or the catheter. Furthermore, releasing the implant is reliable and fast. Further, the risk of deforming the implant and a resulting blocking of the insertion device, as it occurs in case of devices from the prior art which operate with hooks and eyes, can be eliminated. Advantageously, the insertion device can be a catheter. Particularly advantageous, the insertion device can be used for mounting and releasing a prosthesis, a cardiac valve, or a stent.

Moreover, it is proposed that the insertion device has a stopper which limits a movement of the implant in the direction of a proximal end of the insertion device. Such a movement would disadvantageously result in a blocking of the implant. A preferred refinement is that the clamping body rests against the stopper, whereby a position of the clamping body during the movement of the outer insertion element in the direction of the proximal end of the insertion device or the relative movement of the two insertion elements is clearly determined. Furthermore, the stopper can be connected to the clamping body, whereby a shifting of the clamping body relative to the stopper can effectively be prevented. Here, any type of connection considered by the person skilled in the art as being useful such as a force fit, a form fit, or an adhesive bond can be considered. Particularly preferred, the clamping is integrally formed with the stopper, whereby a very stable arrangement is possible. Here, “integrally formed” is to be understood such that the stopper and the clamping body are formed from the same component and/or can be separated from each other only by losing the function of at least one of the components.

According to an advantageous configuration, the implant can be a self-expanding implant, whereby it is able to self-actingly open the clamping body through its radial force. Due to the self-expanding implant, an additional expanding means is not required. Thereby, space and mounting efforts for the latter can advantageously be saved. Hereby it is also possible to configure the insertion device in a less complex manner. However, it is principally also possible to use a balloon-expandable implant. However, for this, the insertion device would have to be suitably adapted, which can be independently solved by the person skilled in the art based on the expertise of the same. Particularly advantageous, the implant is configured without fastening elements, whereby said implant can be shortened with respect to implants of the prior art. This has a positive effect, especially on the patient. Consequently, the insertion device can also be implemented without fastening elements. Hereby, an otherwise necessary connecting of fastening elements, such as hooks and eyes, when mounting the implant onto the insertion device is eliminated, whereby rejects due to incorrect mounting can be reduced in a cost-saving manner. This results primarily in time savings during the preparation of the insertion device in the preparation laboratory.

Furthermore, a method is proposed for producing a clamping body of a releasing device for clamping a medical implant in an insertion device, wherein the implant is released by a relative movement between a first and a second insertion element. The clamping body comprises a proximal end which, in the state of use, is remote from a distal end of the insertion device, and a distal end which, in the state of use, faces toward the distal end of the insertion device. The method includes at least the following steps: providing a hose, expanding the hose at least at one end; and splitting the hose at one end into at least two sheaths parts extending along a main extension of the hose.

With the configuration according to the invention, a method can be implemented by means of which a clamping body of a releasing device can be produced in a user-friendly, reliable and fast manner. The hose preferably, has a substantially round cross-section and/or is formed from a flexible material. Furthermore, the hose is provided in a defined shape which is adapted to at least one region of a contour and/or to a diameter of the implant. In addition, an outer extension or an outer diameter of the hose is adapted to the dimensions of the insertion device such as, in particular, to an inner diameter of the outer shaft. “Splitting” is to be understood here as putting in at least one slot. This is preferably carried out substantially parallel to the main extension of the hose from the expanded end, thus the distal end, up to a position close to the end opposite the expanded end, thus the proximal end. The hose is not separated here into two separate parts, but the at least two sheath parts remain connected at the proximal end via a flange. Here, “substantially parallel” is also to be understood as a deviation of the direction of the slot and the main extension of 30°. A main extension of the hose extends in the axial direction.

In a preferred continuation, a material of the hose is reduced at least at one place so as to allow a compression of the material. Hereby, enclosing the implant can take place without material warpage at a contact place of the sheath parts. This is preferably carried out between expanding and splitting. The place of material reduction is preferably located at the end opposing the expanded end or the proximal end of the hose. The reduction is carried out in particular by introducing an opening substantially perpendicular to the main extension of the hose. Here, “substantially perpendicular” is also to be understood as a deviation of the direction of the opening and the main extension of 30° The opening can particularly advantageously be used as stop point for the splitting or for the slot.

Moreover, a method is proposed for clamping a medical implant by means of a clamping body of a releasing device in an insertion device wherein the implant is released by a relative movement between a first and a second insertion element. The clamping body comprises a proximal end which, in the state of use, is remote from a distal end of the insertion device, and a distal end which, in the state of use, faces toward the distal end of the insertion device. The method includes at least the following steps: Inserting at least one proximal end of the implant from the direction of an expanded end into a surface shell of the clamping body which is formed by at least two sheath parts of the clamping body; enclosing the proximal end of the implant with the surface shell of the at least two sheath parts of the clamping body; and placing the clamping body with the implant into at least one insertion element.

With the configuration according to the invention, a method can be implemented by means of which an implant can be placed and fastened in the insertion device in a user-friendly, precise, and fast manner. During enclosing the implant, preferably, the at least two expanded ends of the sheath parts are slid into each other so that they overlap each other at least in certain areas. This results advantageously in a shape which corresponds to the defined shape of the hose, whereby the clamping body in its clamping position is advantageously adapted to the contour of the implant and has a homogenous contour. Another advantageous aspect of the method is that the implant is pressed together during enclosing with the surface shell of the at least two sheath parts of the clamping body, whereby automatically a diameter reduction of the implant can be carried out with minimal effort by means of the clamping body.

### DESCRIPTION OF THE DRAWINGS

The invention is exemplary explained in more detail hereinafter by means of exemplary embodiments illustrated in the drawings. In the figures, schematically:

FIG. 1 shows a section through an advantageous exemplary embodiment of an insertion device and a releasing device;

FIGS. 2A-E show five stages of the production of a clamping body of the releasing device of FIG. 1;

FIG. 3 shows the clamping body of FIG. 2F in a top view on ends of two overlapping sheath parts of the clamping body;

FIG. 4 shows the insertion device with the clamping body of FIG. 2D in a state prior to placing an implant;

FIG. 5 shows the insertion device and the clamping body of FIG. 4 with the implant being placed;

FIG. 6 shows the clamping body and the implant of FIG. 5 in each case in the compressed state in an outer insertion element of the insertion device; and

FIG. 7 shows a schematic illustration of holding forces of the insertion device, the clamping body and the implant.

### DETAILED DESCRIPTION

In the figures, functionally identical or identically acting elements are in each case numbered with the same reference number. Said figures are schematic illustrations of the invention. They do not image specific parameters of the invention. Furthermore, the figures represent only typical configurations of the invention and are not intended to limit the invention to the illustrated configurations.

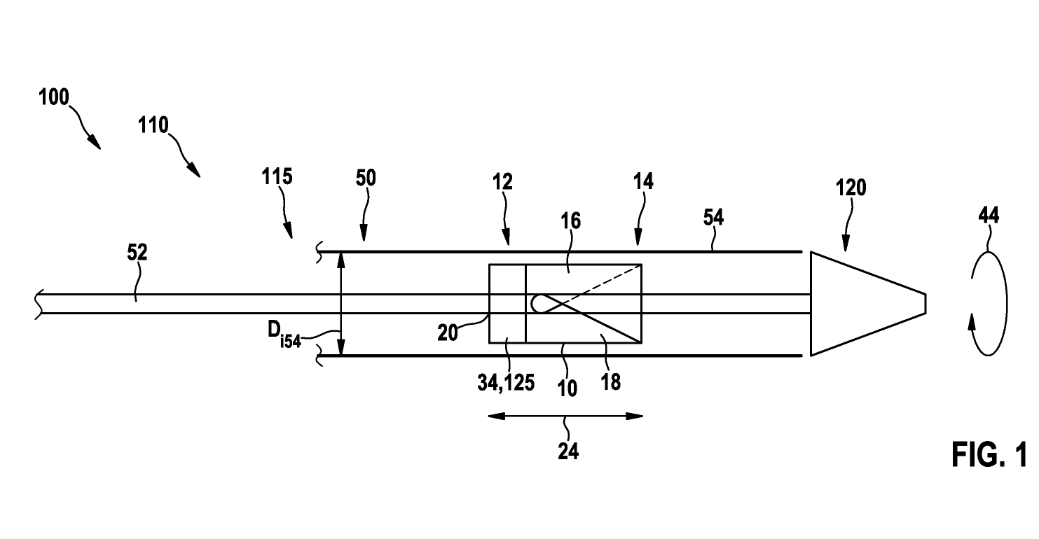

FIG. 1 shows a longitudinal section through an advantageous exemplary embodiment of a releasing device 100 of an insertion device 110 which is only partially illustrated. The insertion device 110 is, for example, a catheter with a shaft region 50 with two coaxially arranged insertion elements 52, 54, e.g., an inner shaft (insertion element 52) and an outer shaft (insertion element 54) surrounding the latter and which, in turn, can be surrounded by a non-shown outer sheath. The insertion device 110 is in user mode, thus faces with its proximal end 115 toward a user when attaching the implant 105 to the releasing device 100 or during the implantation. The implant 105 is placed at the distal end 120 of the shaft region 50 between inner shaft and outer shaft and is to be released at the implantation site in the animal or human body.

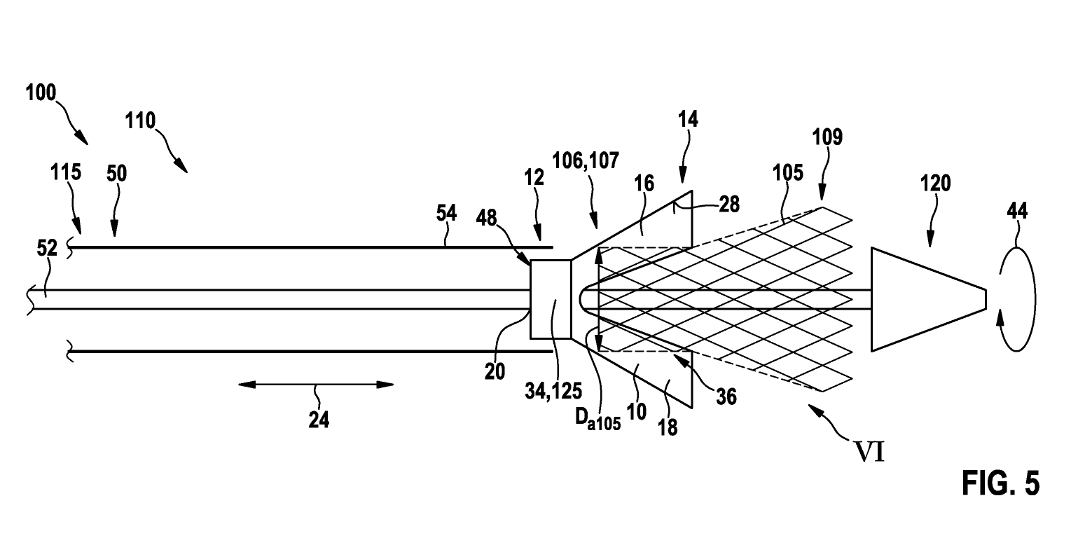

The releasing device 100 serves for detaching the medical implant 105 from the insertion device 110. The implant 105 is arranged at an end 120 of the shaft region 50 which faces away from the user, for example, in proximity to a catheter tip (see FIG. 5). The implant 105 is placed, for example, around the inner insertion element 52 and is released by a relative movement between the first and the second insertion elements 52, 54.

The releasing device 100 comprises a clamping body 10 for clamping the implant 105 in the insertion device 110. In addition, the clamping body 10 has a proximal end 12 which, in the state of use, is remote from the distal end 120 of the insertion device 110, and a distal end 14 which, in the state of use, faces toward the distal end 120 of the insertion device 110.

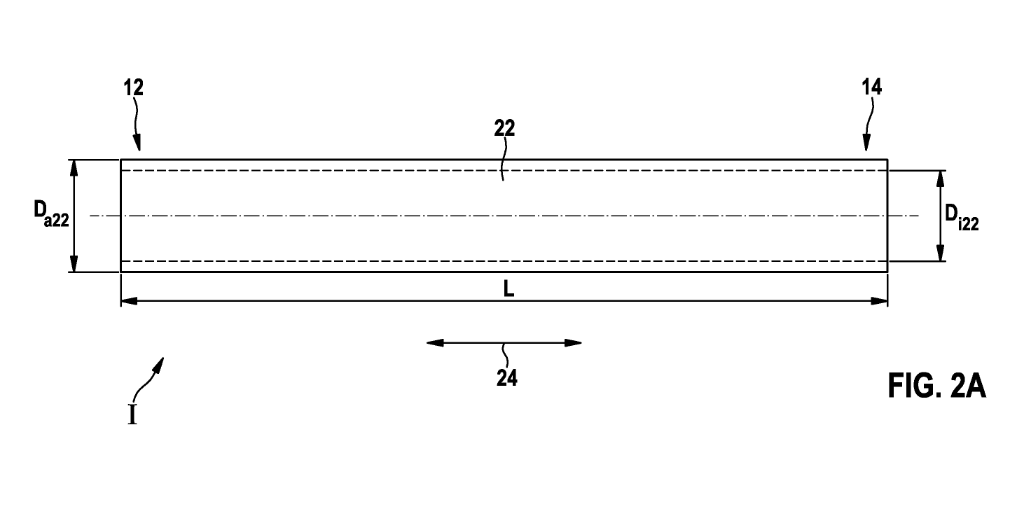

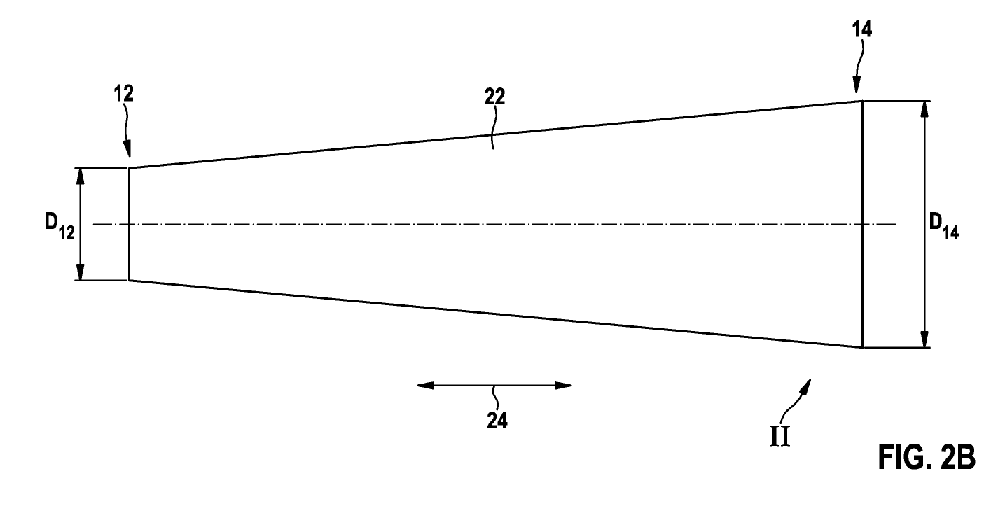

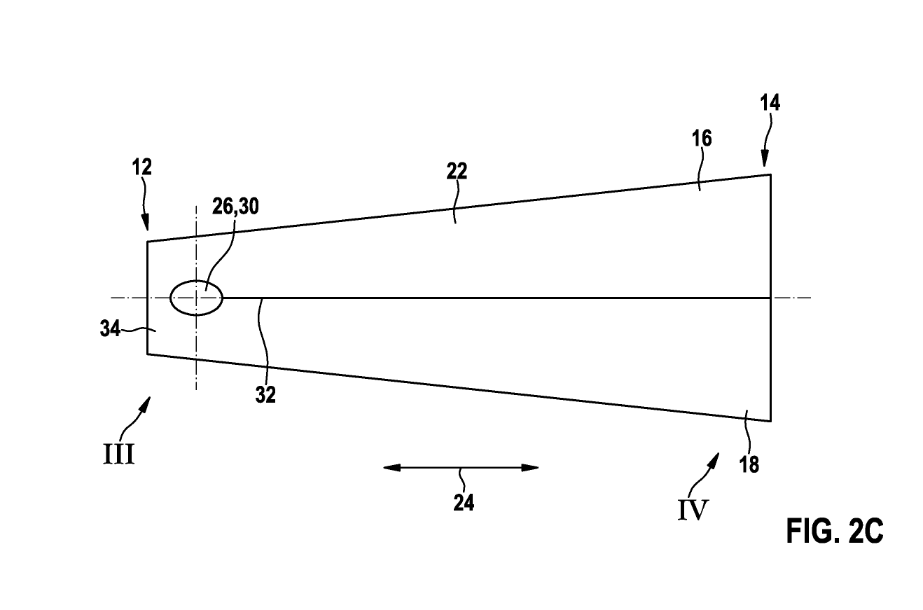

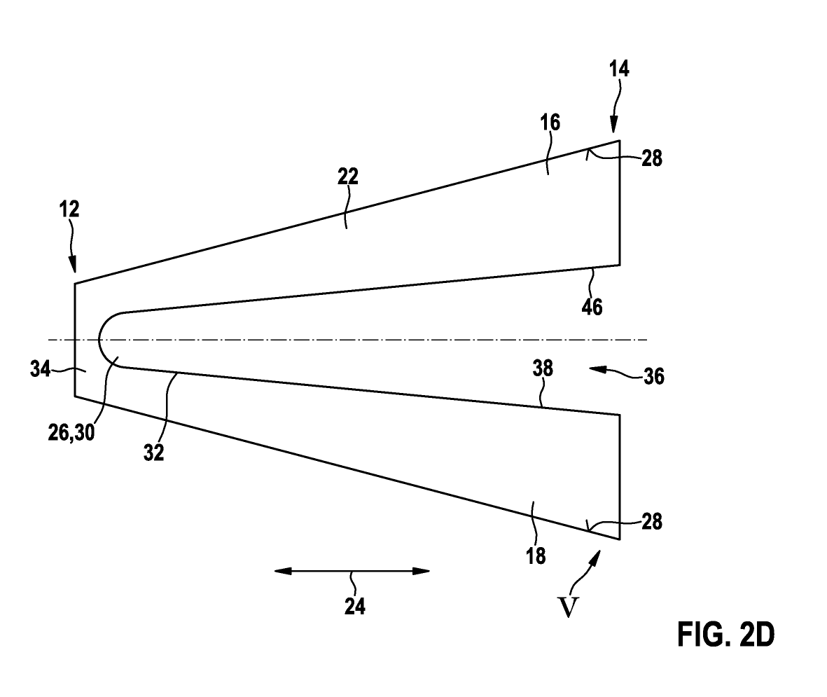

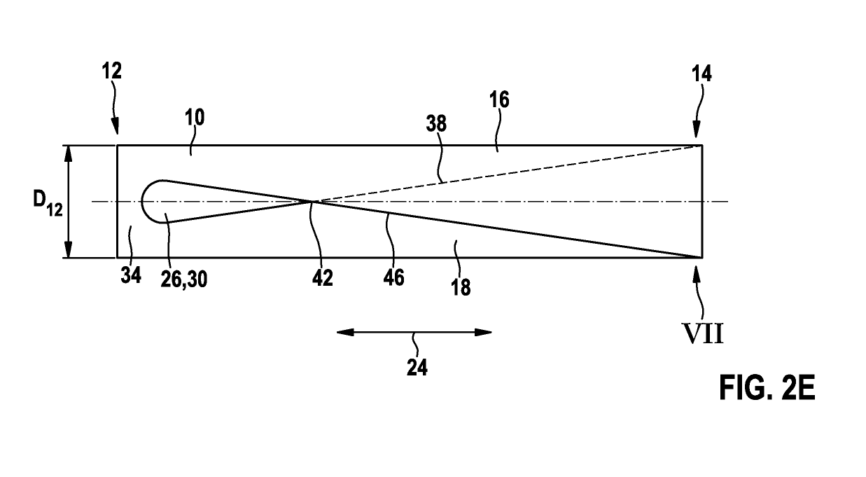

Below, a method for producing the clamping body 10 is described by means of the FIGS. 2A to 2E. As shown in FIG. 2A, a hose 22 is provided (I). This hose 22 is made from a mono-layer design polymer (e.g. PEBAX, of the company Arkema) with high friction, whereby also the clamping body 10 is made from these materials. Alternatively, it would also be possible to produce the hose 22 in a so-called multi-layer co-extruded design with an outer layer and an inner layer. In this case, the outer layer would be made from a material with low friction (e.g. HDPE), and the inner layer would be made from a material with a higher friction (e.g. PEBAX). In addition, the hose 22 has a substantially round cross-section. Further, a dimensioning and an inner diameter Dᵢ₂₂ of the hose are adapted to a contour or an outer diameter Dₐ₁₀₅ of the implant 105 (cf. FIG. 5). Furthermore, these parameters of the hose 22 are adjusted to dimensions of the insertion device 110 such as, for example, an inner diameter Dᵢ₅₄ of the insertion element 54, or the outer shaft. For this, for example, an outer diameter Dₐ₂₂ of the hose 22 can be the inner diameter Dᵢ₅₄ of the outer shaft reduced by 0.2 mm, whereby the implant 105, in the mounted state in the insertion device 110, has to apply a radial force which is sufficient so as to radially push the clamping body 10 by 0.1 mm toward the outer shaft. An inner diameter Dᵢ₂₂, for example, is the outer diameter Dₐ₂₂ of the hose 22 reduced by twice the wall thickness of the hose 22, wherein the wall thickness is, for example, 0.1 mm-0.2 mm. A length L of the hose 22 can be, for example, 15 mm.

In a second step, the hose 22 is expanded at its end 14 with respect to its diameter D₂₂ to a larger diameter D₁₄ (II) (FIG. 2B). In the mounted state of the clamping body 10 in the insertion device 110, this end 14 represents the distal end 14. Expanding can be carried out, for example, by means of a non-shown hot mandrel or a blowing process. In a subsequent third step, shown in FIG. 2C, the material of the hose 22 is reduced at a place 26 (III). This place 26 is arranged at an end 12 which is located opposite to the end 14 and which, in the mounted state of the clamping body 10 in the insertion device 110, represents the proximal end 12. In addition, the material reduction is carried out by means of introducing an opening 30 which extends perpendicular to a main extension 24 of the hose 22 in an axial direction of the hose 22.

In a fourth step, also shown in FIG. 2C, the hose 22 is symmetrically split open at the proximal end 14 (IV), whereby two sheath parts 16, 18 are formed which extend along the main extension 24 of the hose 22. This takes place in that a slot 32 is introduced in the hose 22 from its expanded or distal end 14 parallel to the main extension 24 of the hose 22 up to a position close to the proximal end 12 opposing the expanded end 14. Here, the opening 30 serves as stop point for the splitting or the slot 32. Hereby, the sheath parts 16, 18 are connected at the proximal end 12 via a flange 34.

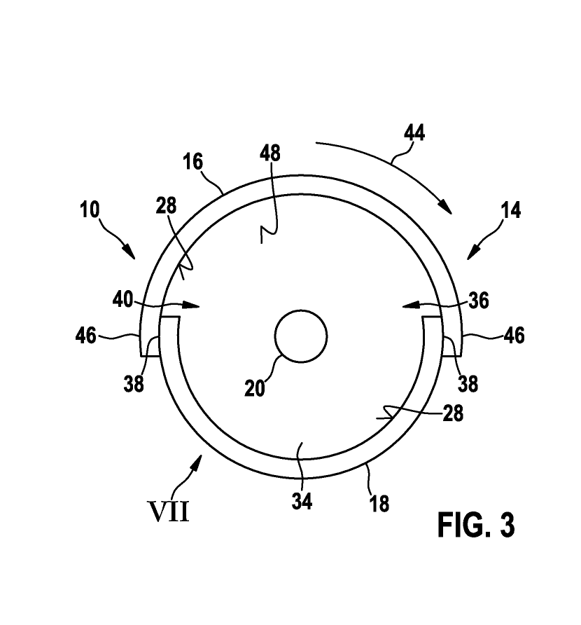

In a fifth step (see FIG. 2D), the hose 22 is opened (V) and thus provides a recess 36 in which the implant 105 can be inserted. The recess 36 is at least partially enclosed by the sheath parts 16, 18 and has a surface shell 28 which therefore is formed by the two sheath parts 16, 18. In this open position, the clamping body 10 is ready to use. In FIG. 2E, the clamping body 10 is shown exemplary in its clamping position without implant 105. In order to reach this position, the sheath parts 16, 18 are compressed against each other, and the edges 38 of the one sheath part 18, which face toward the other sheath part 16, are slid into a lumen 40 of the other sheath part 16. Due to the opening 30, this can be carried out without material warpage at a contact point 42 of the sheath parts 16, 18, whereby the material reduction serves for enabling a compression of the material (VII). Thus, the sheath parts 16, 18 overlap in the circumferential direction 44 at their edges 38, 46. This is shown in particular in FIG. 3 which shows the compressed clamping body 10 in a top view on the distal ends 14 of the overlapping sheath parts 16, 18. Furthermore, in this position, at the distal end 14, the clamping body 10 has again the initial diameter D₂₂ of the hose 22 which also corresponds to a diameter D₁₂ of the clamping body 10 at the proximal end 12. Hereby, the clamping body 10 has a homogenous contour in its main extension 24. A clamping body 10 compressed in this manner can easily be inserted into the insertion element 54 or the outer shaft.

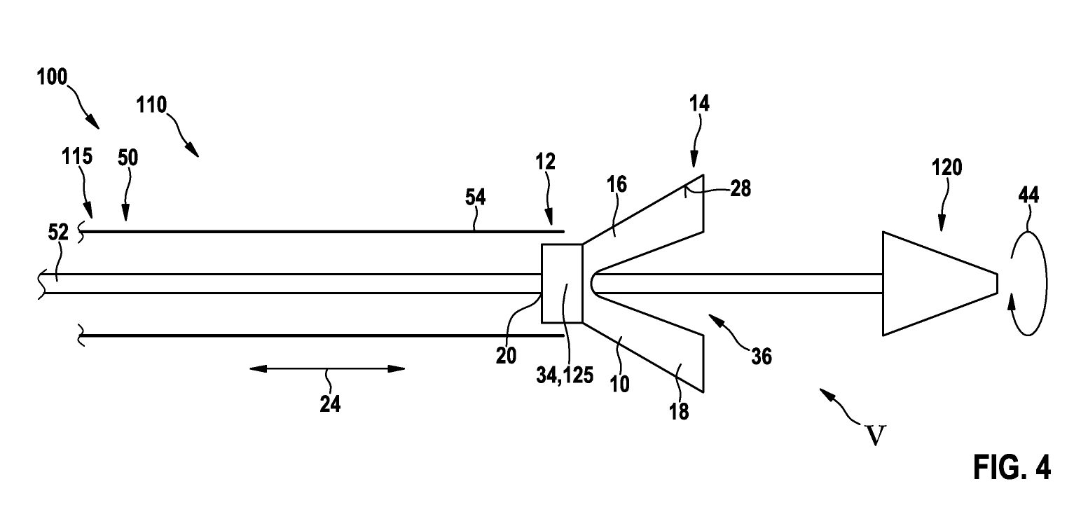

FIG. 1 shows the clamping body 10 in such a compressed position arranged in the insertion device 110. For arranging the clamping body 10 on the insertion element 52 or the inner shaft, the clamping body 10 has a feed-through 20 for the insertion element 52. FIG. 4 shows the insertion device 110 prior to placing the implant 105. For this, the outer insertion element 54 was moved toward the proximal end 115 of the insertion device 110 until the clamping body 10 automatically opens due to a radial force of the implant 105 (V). For limiting the movement of the implant 105 in the direction of the proximal end 115 of the insertion device 110, the insertion device 110 has a stopper 125. This stopper 125 is formed by the flange 34 and thus is formed integrally with the clamping body 10.

FIG. 5 shows the placement of the implant 105 in the opened clamping body 10. The implant 105, for example, a stent or an artificial heart valve implant is implemented in a self-expanding manner and without fastening elements. Below, a method for clamping the implant 105 by means of the clamping body 10 in continuation of the method steps of the production of the clamping body 10 is described by means of the FIGS. 5 to 7.

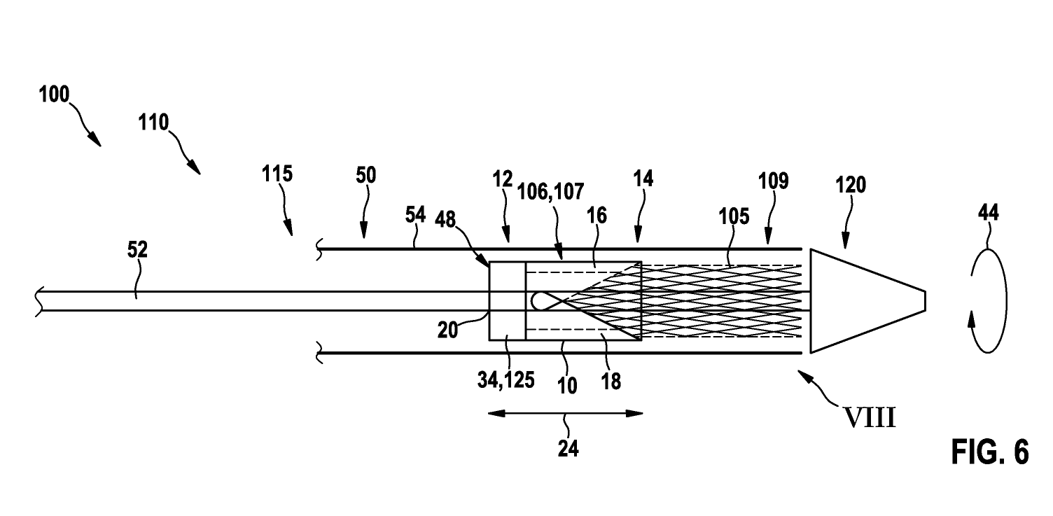

Here, in a first step, a proximal end 107 of the implant 105, which is crimped onto the inner shaft, is inserted with the inner shaft into the surface shell 28 of the recess 36 of the clamping body 10 (VI). This is carried out coming from the direction of the expanded or distal end 14 of the clamping body 10 (FIG. 5). In a subsequent second step, the sheath parts 16, 18 are compressed and thus, the proximal end 107 of the implant 105 is enclosed with the surface shell 28 of the two sheath parts 16, 18 (VII). During enclosing with the surface shell 28 of the two sheath parts 16, 18 of the clamping body 10, the implant 105 is pressed together (VII). Enclosing can be carried out manually or by moving the outer insertion element 54 or the outer shaft toward the distal end 120 of the insertion device 110, whereby the clamping body 10 with the implant 105 is placed in the insertion element 54 (VIII) (see FIG. 6).

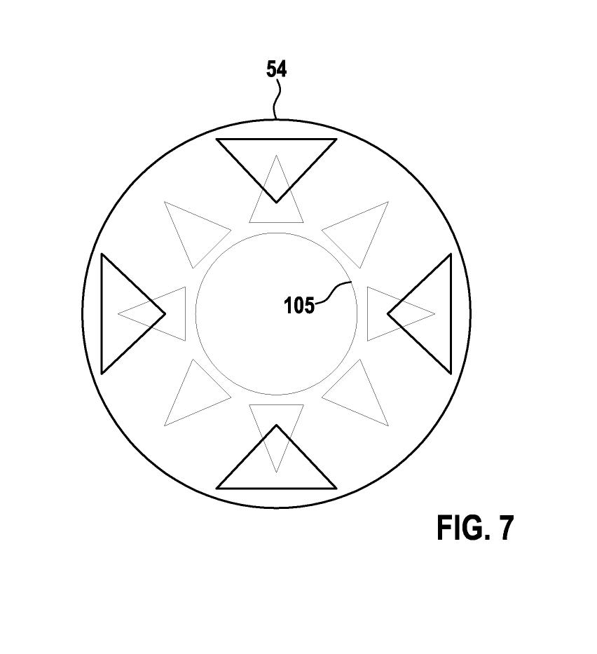

As additionally shown in FIG. 6, the clamping body 10 encloses the implant 105 in the clamping state at a place 106 or the proximal end 107 in a cap-like manner. Here, the sheath parts 16, 18 are arranged in the circumferential direction 44 about the proximal end 107 of the implant 105. Moreover, the flange 34 or the stopper 125 is arranged in a plane 48 which extends perpendicular to the main extension 24 of the clamping body 10. Thus, in the clamping state, the two expanded, partially overlapping sheath parts 16, 18 enclose the proximal end 107 of the implant 105. As illustrated in FIG. 7, in the clamping state, an interaction between a holding force of an insertion element 54 and a radial force of the implant 105 keep the clamping body 10 and the implant 105 in position. In addition, due to its material properties, the clamping body 10 has a high static friction. Due to the static friction between the clamping body 10 and the implant 105, the clamping body 10 has a holding force which, in the clamping state, keeps the implant 105 in the insertion device 110 in position, whereby sliding of the implant 105 is prevented. For the static friction F_{R}, this results in an inequality: F_{R}≦μ_{H}·F_{N}, with μ_{H}: static friction coefficient (material-dependent number), and F_{N}: nominal force resulting from the radial force of the implant 105. Thus, in the clamping state, an interaction between the holding force of the insertion element 54 and the holding force of the clamping body 10 keeps the implant 105 in position.

For implanting the implant 105 in the body, the insertion device 110 prepared in this manner is inserted in the body (not shown). Through the movement of the outer insertion element 54 toward the proximal end 115 of the insertion device 110, a distal end 109 of the implant 105, when being exposed, is first opened and positioned due to its capability of self-expansion (not shown). If the outer shaft is now pulled back to the stopper 125, the sheath parts 16, 18 of the clamping body 10 are exposed, whereby they open automatically due to radial force of the implant 105 (see FIG. 5). Hereby, due to its radial force, the proximal end 107 of the implant 105 is also released and opened. Subsequently, the releasing device 100 or the clamping body 10 is pulled back with the inner shaft into the outer shaft, whereby the clamping body 10 is compressed again (cf. FIG. 1) and removes the insertion device 110 from the body. The implant 105 remains fully positioned in the body.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A releasing device for detaching a medical implant from an insertion device, wherein the implant can be released by a relative movement between a first and a second insertion element, the releasing device comprising a clamping body for clamping the implant in the insertion device, with a proximal end which, in the state of use, is remote from a distal end of the insertion device and a distal end which, in the state of use, faces toward the distal end of the insertion device, wherein in the clamping state, the clamping body encloses the implant at least at one place in a cap-like manner.

2. The releasing device according to claim 1, wherein in the clamping state, two expanded, at least partially overlapping sheath parts of the clamping body enclose at least one proximal end of the implant.

3. The releasing device according to claim 1, wherein in the clamping state, an interaction between a holding force of an insertion element and a holding force of the clamping body keeps the implant in position.

4. The releasing device according to claim 1, wherein the clamping body can be opened due to a radial force of the implant.

5. The releasing device according to claim 1, wherein the clamping body has a feed-through for one of the insertion elements.

6. The releasing device according to claim 1, wherein the clamping body comprises a material with high static friction so as to keep the implant in the clamping state in position.

7. The releasing device according to claim 6, wherein the material is a polymer selected from the group consisting of a polyamide, a polyester, a polyether block amide, silicone, and polyurethane.

8. The releasing device according to claim 1, wherein the clamping body is implemented in a mono-layer design or a multi-layer design.

9. An insertion device for inserting a medical implant which can be released by a relative movement between a first and a second insertion element, the insertion device comprising a releasing device for detaching the medical implant according to claim 1, comprising a clamping device for clamping the implant in the insertion device, with a proximal end which, in the state of use, is remote from a distal end of the insertion device and a distal end which, in the state of use, faces the distal end of the insertion device, wherein in the clamping state, the clamping body encloses the implant at least at one place in a cap-like manner.

10. The insertion device according to claim 9, wherein a stopper is provided which limits a movement of the implant toward a proximal end of the insertion device, wherein the clamping body rests against the stopper or is integrally formed with the stopper.

11. The insertion device according to claim 9, wherein the implant is a self-expanding implant or is implemented without fastening elements.

12. A method for producing a clamping body of a releasing device according to claim 1, for clamping a medical implant in an insertion device, wherein the implant is released by a relative movement between a first and a second insertion element, wherein the clamping body comprises a proximal end which, in the state of use, is remote from a distal end of the insertion device and a distal end which, in the state of use, faces the distal end of the insertion device, including at least the following steps:
providing a hose;
expanding the hose at least at one end; and
splitting the hose at one end into at least two sheath parts extending along a main extension of the hose.

13. The method for producing a clamping body according to claim 12, wherein before the step of splitting the hose, a material of the hose is reduced at least at one place so as to enable compressing the material.

14. A method for clamping a medical implant by means of a clamping body of a releasing device according to claim 1, in an insertion device, wherein the implant is released by a relative movement between a first and a second insertion element, wherein the clamping body comprises a proximal end which, in the state of use, is remote from a distal end of the insertion device and a distal end which, in the state of use, faces toward the distal end of the insertion device, including at least the following steps:
inserting at least one proximal end of the implant coming from the direction of an expanded end into a surface shell of the clamping body, which is formed from at least two sheath parts of the clamping body;
enclosing the proximal end of the implant with the surface shell of the at least two sheath parts of the clamping body; and
placing the clamping body with the implant in at least one insertion element.

15. The method for clamping a medical implant according to claim 14, wherein during enclosing with the surface shell of the at least two sheath parts of the clamping body, the implant is pressed together.
